(19) United States
(12) Patent Application Publication
(10) Pub. No.: US 2025/0235288 A1
(43) Pub. Date: Jul. 24, 2025

(54) CAMERA-BASED GUIDANCE FOR NEEDLE INTERVENTION

(71) Applicant: Siemens Healthineers AG, Forchheim (DE)

(72) Inventors: Christian HOPFGARTNER, Fuerth (DE); Andreas WIMMER, Forchheim (DE); Michael SUEHLING, Erlangen (DE)

(73) Assignee: Siemens Healthineers AG, Forchheim (DE)

(21) Appl. No.: 19/032,888

(22) Filed: Jan. 21, 2025

(30) Foreign Application Priority Data
Jan. 23, 2024 (EP) 24153415.5

(51) Int. Cl. A61B 90/00; A61B 34/10

(52) U.S. Cl. CPC A61B 90/361; A61B 34/10; A61B 2034/107

(57) ABSTRACT

A method for generating augmented camera image data for guiding a needle intervention, comprises: receiving medical image data and camera image data of an examination portion of a patient; determining information concerning a planned insertion pose of the needle based on the medical image data, the information including coordinates assigned to the planned insertion pose of the needle in a coordinate system of the medical image data; determining coordinates assigned to the planned insertion pose of the needle in the camera coordinate system of the camera by transforming the coordinates assigned to the planned insertion pose from the medical image data coordinate system into the camera coordinate system; and generating augmented camera image data based on the camera image data and the coordinates in the camera coordinate system.

### CROSS-REFERENCE TO RELATED APPLICATION(S)

The present application claims priority under 35 U.S.C. § 119 to European Patent Application No. 24153415.5, filed Jan. 23, 2024, the entire contents of which is incorporated herein by reference.

### FIELD

One or more example embodiments of the present invention relate to a method for generating augmented camera image data for guiding a needle intervention. One or more example embodiments of the present invention also concern a camera-based method for guiding a needle intervention. Further, one or more example embodiments of the present invention refer to an intervention control device. Furthermore, one or more example embodiments of the present invention relate to an intervention system.

### BACKGROUND

Medical imaging systems are also often used to guide needle interventions like biopsies. During preparation of the intervention, a needle path is planned based on a planning scan, e.g. a CT spiral scan. The planning consists of annotating a target point inside the body where the needle tip shall be positioned, and a needle insertion point on the skin surface. The needle path is planned such that sensitive structures like vessels, other organs, or bones are not harmed. The key challenge in intervention workflows is to transfer the needle path planned based on the medical data to the actual patient. In particular, the exact insertion point on the skin has to be located and the correct direction has to be applied when inserting the needle into the body.

For identification of the needle entry point on the patient's skin, the following workflow is most common: the z-position of the slice (the z-axis is the symmetry axis of the medical imaging system) in which the entry point is located is read from the image text of the planning scan. The patient table is positioned in a way that this z-position exactly lines up with the scan plane, which is marked by the gantry laser. In this way, the z-position for the injection point is located. To identify the x- and y-position within the z-plane, the distance on the skin from the central laser is measured within the planning image and is also measured on the actual patient with a ruler and marked on the skin. As an alternative for this measurement, a metal grid can be used which is taped to the skin before the planning scan and which has metal wires running parallel in z-direction.

The wires appear on the planning scan and can be used to identify the ideal injection point (e.g. between the 3rd and 4th wire from the left). When the injection point has been located and marked on the patient's skin using one of the two methods described above, the needle must be inserted into the skin with the correct angle as planned in the planning scan. The user views the planned needle path on an in-room monitor next to the patient and aligns the angle in which he injects the needle into patient's body with the angle of the planned needle path using an eye balling approach. If the needle path is only tilted within the x-y scan plane (in-plane intervention) this method works relatively well. However, if the needle is also tilted towards the z-axis (double angulated needle path) this is not easy to accomplish by eyeballing resulting in a decreased precision. As an alternative, few radiologists actually measure the angle of the needle to match the angle on the planned needle path.

### SUMMARY

It is therefore a task to provide a method and a system for guiding a needle intervention with more reliability than in prior art.

This task is accomplished by a method for generating augmented camera image data for guiding a needle intervention according to claim 1, by a camera-based method for guiding a needle intervention according to claim 11, by an intervention control device according to claim 12 and by an intervention system according to claim 13.

The method for generating augmented camera image data for guiding a needle intervention according to an embodiment of the present invention comprises the step of receiving medical image data of an examination portion of a patient. The examination portion comprises a region of the body of a patient which is intended to be the object of a medical intervention performed using a needle. The medical image data are generated by a medical imaging system. The medical imaging system comprises an imaging technique for generating 3D medical images of the inside of the body of a patient. The medical imaging system preferably comprises an X-ray based imaging system. Most preferably, the medical imaging system comprises a computer tomography system. However, the medical imaging may alternatively comprise other modalities, e.g. a magnetic resonance or ultrasound imaging system. The medical image data preferably comprise 3D images. The medical image data provide information for determining and planning a pose of a needle for a planned intervention. In particular, the medical image data comprise information about the interior of the body of a patient and details of the examination portion such that sub-portions of the inner body which have to be avoided by the needle and sub-portions of the inner body which are intended to be drilled through by the needle are able to be identified and to be localized. The medical image data are assigned to a coordinate system of the medical image data. Such a medical image data coordinate system preferably comprises a 3D-coordinate system including the origin on a symmetry axis, in particular the z-axis of the medical imaging system.

Further, camera image data of the examination portion are received. The camera image data are generated by an observation camera or monitoring camera being arranged in the examination room for observing the patient lying on the patient table of the medical imaging system. Preferably, the camera comprises a 2D image camera. 2D images enable a complete overview of an insertion scenario. The camera image data are intended to be used for monitoring and guiding a needle intervention in an examination portion of a patient. Generally, the coordinate system of the camera, also named camera coordinate system, differs from the coordinate system of the medical imaging system.

Furthermore, information concerning a planned insertion pose of the needle is determined based on the received medical image data as already mentioned above. The information about the planned insertion pose of the needle comprises at least information about a planned position of the needle, in particular of a planned insertion position of the needle and an orientation of the needle while the needle is inserted. Further, the information concerning a planned insertion pose of the needle preferably comprises information about the planned end position of the tip of the needle and also preferred information about the planned end position of the head of the needle and further preferred information about the planned orientation of the needle at the end position.

For determining the mentioned information, coordinates assigned to the planned insertion pose of the needle in the medical image data coordinate system are determined based on the medical image data. In particular, the medical image data provide information about organs or bones or tissue or blood vessels to be targeted in the body of a patient using the needle and organs or bones or tissue or blood vessels to be avoided by the needle.

Furthermore, coordinates assigned to the planned insertion pose of the needle in the camera coordinate system of the camera are determined by transforming the coordinates assigned to the planned insertion pose of the needle from the medical image data coordinate system into the camera coordinate system. The coordinate system of the medical imaging system and the coordinate system of the camera differ from each other due to different positions of the origins of these different systems, due to different orientation of these systems and possibly due to a different geometry of these systems. Based on the medical image data, a planned pose of the needle is able to be determined. However, since the template for the needle is to be shown in the display of the camera, a transformation between the coordinate systems of these different systems has to be performed.

In the end, augmented camera image data are generated based on the determined coordinates in the camera coordinate system and the received camera image data by combining templates or marks based on the determined coordinates with the received camera image data.

Using a camera, the workflow step of transferring a planned needle path to the actual patient can be simplified significantly. Many modern medical imaging systems are already equipped with cameras for patient observation or for patient positioning and scan automation. Moreover, observation cameras are also frequently installed in examination rooms. The use of an already existing camera in the context of an imaging-guided medical intervention workflow, particularly for the initial placement of the needle, provides a precise placement of the needle based on a path planned using the medical imaging data. The display of the needle tip may help to save time and to avoid mistakes during the otherwise manual transfer of the medical imaging planning result to the skin of the patient. In addition, having a good indication of the planned needle orientation and stop position may help to avoid some control scans during the intervention to track the needle, thereby saving time and radiation dose. In contrast to alternative solutions, there is no need for additional resources, as already existing and installed hardware is used for observing the needle.

In the camera-based method for guiding a needle intervention according to an embodiment of the present invention, the method for generating augmented camera image data for guiding a needle according to an embodiment of the present invention is performed, wherein coordinates assigned to a planned insertion pose of the needle in a camera coordinate system of a camera are determined and augmented camera image data are generated based thereon. The augmented camera image data preferably comprise templates or marks as needle alignment guide for guiding an insertion of the needle. Preferably, the templates or marks comprise a guideline, preferably comprising at least one of the following signs: a dotted line, a solid line, a marking cross, a marking circle. Further, the augmented camera image data are displayed.

Hence, the needle and a planned pose of the needle are displayed in the camera image data based on the determined coordinates. The camera-based method for guiding a needle intervention according to an embodiment of the present invention shares the advantages of the method for generating augmented camera image data for guiding a needle intervention.

The intervention control device according to an embodiment of the present invention comprises a medical image data interface for receiving medical image data of an examination portion of a patient and a camera image data interface for receiving camera image data of the examination portion.

Further, the intervention control device according to an embodiment of the present invention comprises a determination unit for determining information concerning a planned insertion pose of a needle based on the received medical image data. For determining information concerning a planned insertion pose of the needle, coordinates assigned to the insertion pose of the needle in the medical image data coordinate system are determined based on the medical image data.

Furthermore, the intervention control device according to an embodiment of the present invention comprises a transformation unit for determining the coordinates assigned to the insertion pose of the needle in the camera coordinate system of the camera by transforming the coordinates assigned to the insertion pose in the medical image data coordinate system into the camera coordinate system.

The intervention control device also comprises a generation unit for generating augmented camera image data based on the determined coordinates in the camera coordinate system and based on the received camera image data.

The intervention control device according to one or more embodiments of the present invention shares the advantages of the camera-based guidance method for guiding a needle intervention according to one or more embodiments of the present invention.

The intervention system according to an embodiment of the present invention comprises a medical imaging system, preferably a CT-system, for generating medical image data from an examination portion of a patient. Further, the intervention system comprises a camera for observing the patient and for acquiring camera image data from the patient and in particular from the examination portion of the patient. Furthermore, the intervention system includes a needle for an intervention.

The intervention system also comprises an intervention control device according to an embodiment of the present invention for generating augmented camera image data based on medical image data from the medical imaging system and camera data from the camera and for controlling the intervention using the needle based on the augmented camera image data.

The intervention system further comprises a display for displaying the augmented camera image data generated by the intervention control device. In the augmented camera image data, the needle and a template or mark indicating a planned pose of the needle are depicted. Hence, the display is used for displaying the camera image data and for displaying a template of the planned pose of the needle such that a physician is enabled to follow the displayed template with the needle. The intervention system shares the advantages of the intervention control device according to one or more embodiments of the present invention.

Some units or modules of the intervention control device mentioned above can be completely or partially realized as software modules running on a processor of a respective computing system, e.g. of a control device of a medical imaging system. A realization largely in the form of software modules can have the advantage that applications already installed on an existing computing system can be updated, with relatively little effort, to install and run these units of the present application. The object of one or more embodiments of the present invention is also achieved by a computer program product with a computer program that is directly loadable into the memory of a computing system, and which comprises program units to perform the steps of the inventive method for generating augmented camera image data for guiding a needle intervention, in particular the steps of receiving medical image data, of receiving camera image data, of determining information concerning a planned insertion pose of the needle, of determining coordinates assigned to the planned insertion pose of the needle in the camera coordinate system and of generating augmented camera image data, or the software-based steps of the camera-based method for guiding a needle intervention, in particular the above-mentioned steps of the method for generating augmented camera image data for guiding a needle intervention, when the program is executed by the computing system. In addition to the computer program, such a computer program product can also comprise further parts such as documentation and/or additional components, also hardware components such as a hardware key (dongle etc.) to facilitate access to the software.

A non-transitory computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

The dependent claims and the following description each contain particularly advantageous embodiments and developments of the present invention. In particular, the claims of one claim category can also be developed analogously to the dependent claims of another claim category. In addition, within the scope of the present invention, the various features of different exemplary embodiments and claims can also be combined to form new exemplary embodiments.

In a preferred variant of the method for generating augmented camera image data for guiding a needle intervention according to an embodiment of the present invention, the information concerning a planned insertion pose of the needle in the received medical image data comprises an insertion point of the needle. Advantageously, the planned insertion point is displayed as template such that a physician is enabled to guide the needle to the virtually marked insertion position visible on a display.

Assuming that the patient has not moved since the planning scan was performed, the needle insertion point qCT from the planning scan, i.e. the medical image data in the medical image data coordinate system, can be transferred to the camera image point qCAM in the 3D camera coordinate system using the scanner to camera transform TCT, CAM:

$${q}_{C A M} = {T}_{CT , CAM} \cdot {q}_{C T} . \qquad ( 1 )$$

In case at the intervention treatment that table position is different to the planning scan position, an additional translation regarding the new table position (describing by the transformation TTABLE) has to be applied while computing the point in camera coordinates:

$${q}_{C A M} = {T}_{CT , CAM} \cdot {T}_{TABLE} \cdot {q}_{C T} . \qquad ( 2 )$$

The needle insertion point in camera coordinates qCAM is then projected into the camera image data using the intrinsic calibration expressed through the projection PCAM:

$${q}_{2 D} = {P}_{C A M} \cdot {q}_{C A M} . \qquad ( 3 )$$

The intrinsic calibration of the camera consists of the focal length f, the principal point, and distortion coefficients. A visual marker like a dot is displayed as an overlay onto the camera image in the camera image data. The physician can then align the tip of the actual needle with the marker to insert the needle at the right position.

As discussed above, preferably, the sub-step of transforming the coordinates assigned to the insertion pose in the medical image data coordinate system into a 3D camera coordinate system comprises an application of a camera transform TCT, CAM to the insertion pose assigned to the medical image data coordinate system. Advantageously, the coordinates are transformed into the coordinate system of the camera which is used for observing the needle and in particular the insertion of the needle.

As also mentioned above, preferably, the sub-step of transforming the coordinates assigned to the insertion pose in the medical image data coordinate system into a 3D camera coordinate system comprises an application of a transformation TTABLE representing a new table position in case the table position is changed. Advantageously, a movement of the table is taken into account for the coordinates in the camera image.

In a preferred version of the method for generating augmented camera image data for guiding a needle intervention according to an embodiment of the present invention, the camera transform TCT, CAM is determined based on an extrinsic calibration step. Advantageously, the camera transform TCT, CAM can be calculated based on extrinsic calibration parameters. Extrinsic parameters describe the position and orientation of a camera in space.

Preferably, the camera image data comprise 2D camera image data as discussed later. Advantageously, 2D image data can be simply acquired and easily perceived by a physician, wherein the physician gets a complete overview of a needle insertion scenario.

Preferably, the information concerning a planned insertion pose of the needle based on the received medical image data comprises a needle orientation for the needle intervention and the step of determining information concerning an insertion pose of the needle in the received medical image data comprises at least one of the following sub-steps, preferably more than one of the following sub-steps, most preferably all of the following sub-steps, of determining:
an end point of the needle at the start of the insertion based on a target needle tip position,
a planned needle direction and
the length of the needle.

Hence, the augmented-reality visualization is further enriched with information about the orientation in which the needle shall be inserted. Advantageously, the physician receives all the information about the planned pose of the needle to be inserted into the examination portion.

For calculating an end point of the needle, the knowledge about the length 1 of the needle is used. Then the needle end point uCT in the medical image data coordinate system is constructed at the start of the insertion by virtually moving the needle insertion point qCT in opposite direction of the planned needle direction. The planned needle direction dCT from the needle insertion point q to the planned needle end point is calculated as follows:

$${d}_{C T} = ( {p}_{CT} - {q}_{C T} ) / ‖ {p}_{CT} - {q}_{C T} ‖ , \qquad ( 4 )$$

wherein dCT is normalized and pCT is the target needle tip position inside the body of a patient in the medical image data coordinate system.

The needle end point uCT is calculated as follows:

$${u}_{C T} = {q}_{C T} - l {d}_{CT} , \qquad ( 5 )$$

where dCT is normalized. This needle end point uCT is then also transformed to camera coordinates uCAM and preferably projected into the 2D camera image, wherein the needle end point u2D in the 2D camera image is calculated as follows:

$${u}_{2 D} = {P}_{CAM} {u}_{CAM} . \qquad ( 6 )$$

The needle insertion point q and the end point u are visualized for example as dots or as line ranging from q2D to u2D in a live camera image, preferably a 2D live camera image.

Hence, also preferred, the needle end point uCT at the start of the insertion is determined by virtually moving the needle insertion point qCT in the opposite direction of the planned needle direction, as explained in context with formula (4). Advantageously, a second marking point for positioning the needle at the start of the insertion is determined which facilitates the positioning and alignment of the needle at the beginning of the insertion.

As mentioned above, the information concerning an insertion pose of the needle in the received medical image data preferably comprises an end point of the needle head at the end of the needle intervention. Advantageously, the physician receives a precise information about the end position of the needle head he has to reach in the end of the insertion.

In case the information concerning a planned insertion pose comprises an end point of the needle head at the end of the needle intervention, preferably, the end point of the needle head at the end of the needle intervention is determined by adding the target needle tip position to the end point of the needle at the start of the insertion and subtracting therefrom the insertion point of the needle.

The end point uCT of the needle head (at the start of the intervention) is moved in needle direction to the end point vCT of the needle head at the end of the needle intervention:

$${v}_{C T} = {u}_{C T} + {p}_{CT} - {q}_{CT} , \qquad ( 7 )$$

wherein the needle tip is transferred to the target needle tip position pCT inside the body.

If the camera image data comprise 2D image data, a projection into the view plane of the camera is performed as explained above for determining a 2D projection v2D of the end point vCT of the needle head at the end of the needle intervention in the 2D image data:

$${v}_{2 D} = {P}_{C A M} {T}_{C T , C A M} {v}_{C T} . \qquad ( 8 )$$

A visual indication like a dot is then placed at this position to indicate the stop position for the needle end point in the live 2D camera image.

Hence, if the camera image data comprises 2D camera image data, the step of determining the coordinates assigned to the insertion pose of the needle in the camera image data preferably includes the sub-steps of:
transforming the coordinates qCT, uCT, VCT assigned to the insertion pose from the medical image data coordinate system into a 3D camera coordinate system,
projecting the transformed coordinates qCAM, uCAM, VCAM from the 3D camera coordinate system into 2D camera image data, wherein 2D coordinates q2D, u2D, 2D in the 2D camera image data concerning the insertion pose are determined.

Advantageously, the target points of the needle are projected into 2D camera image data, which are used for observing the patient and in particular the examination portion.

Even if the camera image data comprises 3D camera image data, in the method for generating augmented camera image data for guiding a needle intervention according to an embodiment of the present invention, the sub-step of projecting the coordinates concerning the planned insertion pose into the camera image data preferably comprises the step of applying a projection TCT, CAM to the coordinates concerning the insertion pose in the 3D camera coordinate system.

### BRIEF DESCRIPTION OF THE DRAWINGS

The present invention is explained below with reference to the figures enclosed once again. The same components are provided with identical reference numbers in the various figures.

The figures are usually not to scale.

FIG. 1 shows a schematic illustration of an intervention using a needle guided by a laser beam according to prior art,

FIG. 2 shows a schematic illustration of an intervention using a needle guided by a camera of a smartphone according to prior art,

FIG. 3 shows a perspective view of a perspective projection of points in 3D-space onto an image plane,

FIG. 4 shows a sectional view of a perspective projection of points in 3D-space onto the image view plane of a camera,

FIG. 5 shows a 2D view of projected points in the image view plane of a camera,

FIG. 6 shows a virtual image of a needle at the beginning of a treatment,

FIG. 7 shows a virtual image of a needle, when the needle is partially inserted,

FIG. 8 shows a virtual image of a needle including the planned needle end position,

FIG. 9 shows a flow chart illustrating the camera-based method for guiding a needle intervention according to an embodiment of the present invention,

FIG. 10 shows a schematic view of an intervention control device according to an embodiment of the present invention,

FIG. 11 shows an intervention system according to an embodiment of the present invention.

### DETAILED DESCRIPTION

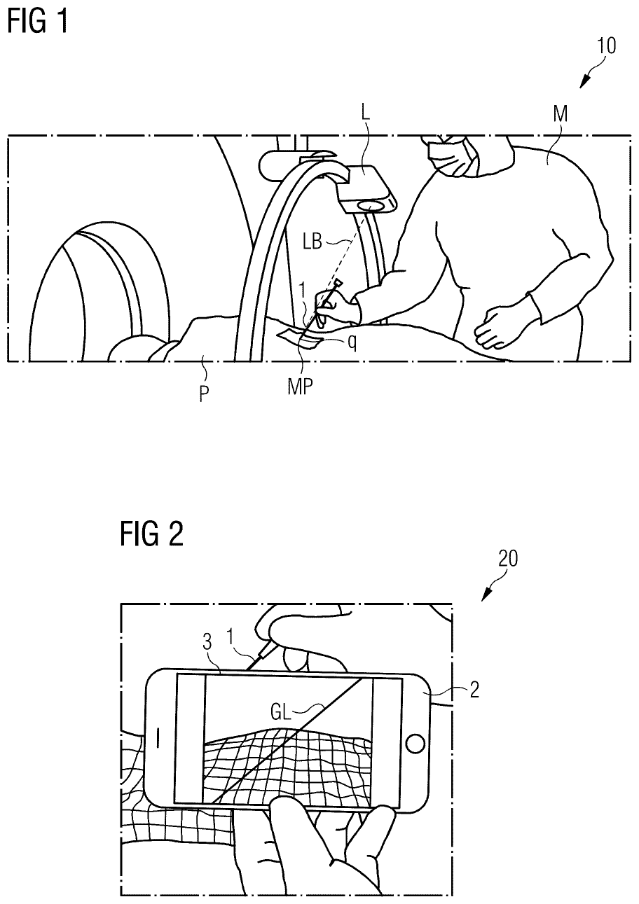

In FIG. 1, a schematic illustration 10 of an intervention using a needle 1 guided by a laser beam LB of a laser L according to prior art is shown. As can be taken from FIG. 1, the initial needle placement on a planned path is assisted by a laser L emitting a laser beam LB onto a patient P, wherein a marking point MP is projected onto the planned insertion position q of the needle 1. Hence, a physician M places the needle 1 on the marked insertion point q guided by the laser beam LB.

In FIG. 2, a schematic illustration 20 of an intervention using a needle 1 guided by a camera of a smartphone 2 is depicted. A smartphone application overlays the planned angle on the smartphone's camera display 3 in real-time based on the smartphone's orientation. The needle's angle is selected by visually comparing the actual needle with the guideline GL in the display 3 of the smartphone 2. However, this system also lacks integration with the CT system.

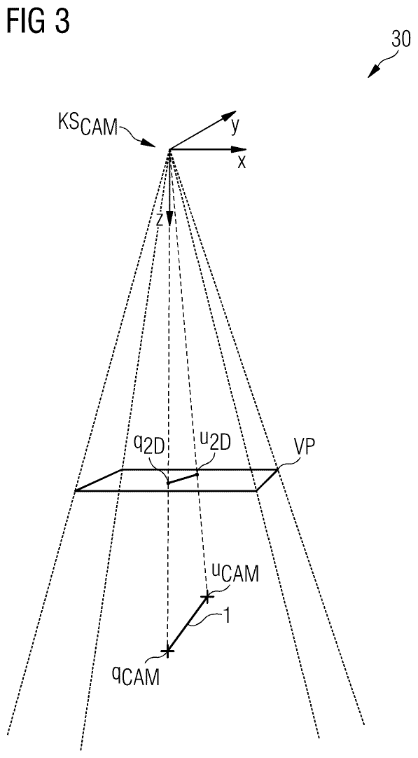

In FIG. 3, a perspective view 30 of a perspective projection of points q, u in 3D-space onto an image plane or view plane VP is shown. In the upper portion of FIG. 3 a coordinate system KSCAM of the camera is depicted, wherein the origin of the coordinate system represents the camera origin. Further, in the view plane VP of the camera a projection of the needle 1 and particularly the projected insertion point q2D and the projected end point u2D of the needle 1 are illustrated. Further, on the lower part of FIG. 3 the needle 1 itself in 3D space is illustrated and the insertion point qCAM and the endpoint uCAM of the needle 1 at the start of the intervention are depicted.

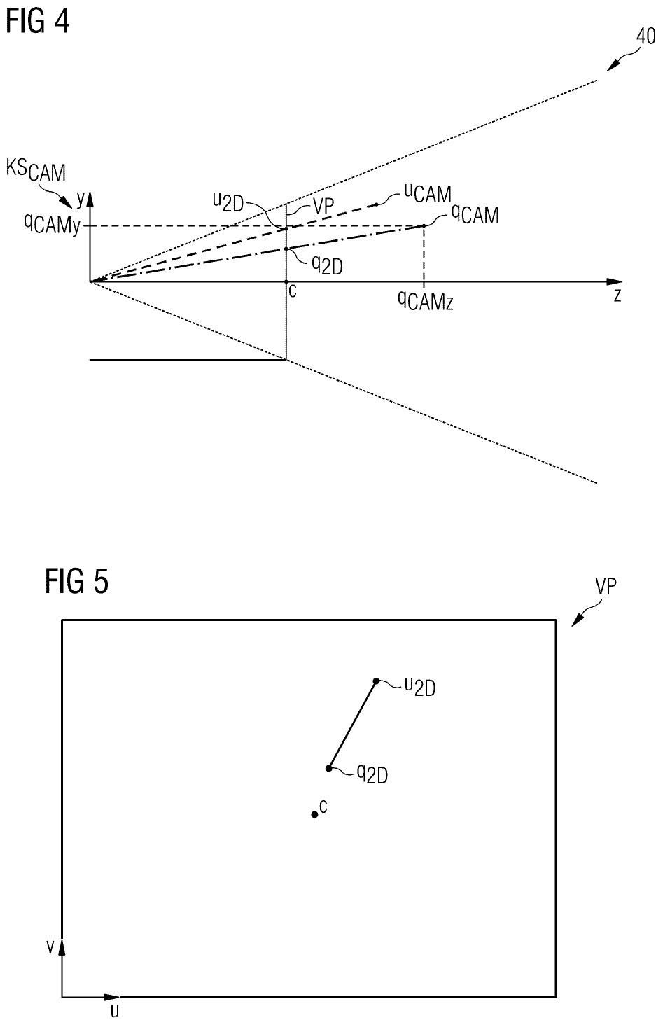

In FIG. 4, a sectional view 40 of a perspective projection of points in 3D-space onto the image view plane VP is shown. As can be taken from the radiation theorem, the v-coordinate q2Dv of the projection q2D of the insertion point is achieved by:

$$q 2 Dv = fy ⋆ qCAMy / qCAMz + cv , \qquad ( 9 )$$

wherein fy is the focal length in y-direction, qCAMy is the y-coordinate of the insertion point qCAM in the 3D camera coordinate system KSCAM, qCAMz is the z-coordinate of the insertion point qCAM in the 3D camera coordinate system KSCAM, cv is the v-coordinate of the intersection point c of the z axis of the 3D camera coordinate system with the view plane VP of the camera. In an analogue manner, also the v-coordinate u2Dv of the projection u2D of the end point u at the start of an intervention can be calculated.

Analogue to formula (9) also the u-coordinate q2Du of the projection q2D of the insertion point q can be calculated as follows:

$$q 2 Du = fx ⋆ qCAMx / qCAMz + cu , \qquad ( 10 )$$

wherein fx is the focal length in x-direction, qCAMx is the x-coordinate of the insertion point qCAM in the 3D camera coordinate system KSCAM, qCAMz is the z-coordinate of the insertion point qCAM in the 3D camera coordinate system KSCAM, cu is the u-coordinate of the intersection point c of the z axis of the 3D camera coordinate system with the view plane VP of the camera.

In FIG. 5, a 2D view of the projected insertion point q2D and the projected end point u2D at the start of the intervention in the image plane VP is illustrated. As can be taken from FIG. 5, in the center of the view plane VP, the intersection point c of the z axis of the 3D camera coordinate system with the view plane VP of the camera is marked. Further the u-axis u and the v-axis v of the coordinate system of the view plane VP is marked on the lower left portion of FIG. 5.

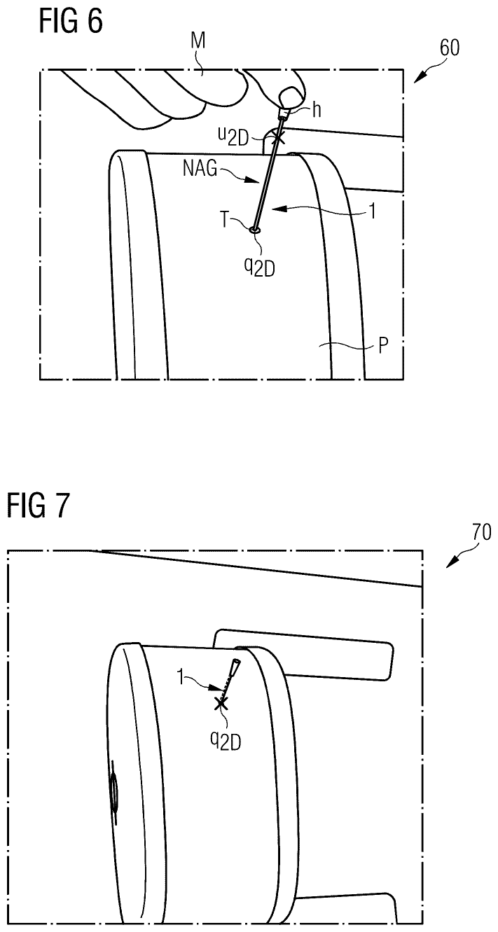

In FIG. 6, a virtual image 60 of a needle 1 at the beginning of a treatment is shown. The needle alignment guide NAG, i.e. the initial needle orientation, is drawn as a solid line. The user needs to align the real needle tip T with one ending of the virtual guidance needle overlay and the needle head h with the other ending of the virtual needle.

The visualization q2D of the needle insertion point q is achieved as follows: in order to provide augmented-reality guidance to the physician M during the intervention, a real-time view of the patient P acquired with the camera is displayed on a screen, for example on an in-room monitor or a tablet next to the patient P. In addition, the planned needle insertion point q is displayed as an overlay graphic, as illustrated in FIG. 6. Therefore, the transformation TCT, AM from scanner/medical image coordinates to camera coordinates is determined in an extrinsic calibration step. This step is performed once during installation of the camera. The calibration can be performed for example with a checkerboard phantom that is aligned to the scanner positioning lasers.

Assuming that the patient P has not moved since the planning scan was performed, the insertion point qCT from the planning scan can be transferred to the camera image point qCAM using the scanner to camera transform TCT, CAM as mentioned in formula (1).

In case at the intervention treatment that table position is different to the planning scan position, an additional translation regarding the new table position (described by the transformation TTABLE) has to be applied while computing the point in camera coordinates according to formula (2).

The needle insertion point in camera coordinates qCAM is then projected into the camera image using the intrinsic calibration expressed through the projection PCAM according to formula (3).

The intrinsic calibration of the camera consists of the focal length f, the principal point, and distortion coefficients. A visual marker like a dot is displayed as an overlay onto the live camera image. The physician can then align the tip of the actual needle with the marker to insert the needle at the right position.

In FIG. 7, a virtual image 70 of a needle 1 when the needle 1 is partially inserted, is shown. The projected insertion point q2D is visualized as a cross.

The augmented-reality visualization may be further enriched with information about the orientation in which the needle 1 shall be inserted. Therefore, the length 1 of the needle 1 is entered into the system. The system then constructs the needle end point uCT at the start of the insertion by moving the needle insertion point qCT in opposite direction of the planned needle direction. The direction dCT from the needle insertion point q to the planned needle end point is calculated according to formula (4).

The needle end point uCT is calculated according to formula (5).

This needle end point uCT is then also transformed to camera coordinates uCAM and projected into the 2D camera image according to formula (6).

The needle insertion point q and end point u are visualized for example as dots or as line ranging from q2D to u2D in the live 2D camera image, as illustrated in FIG. 6. At the start of the intervention, the physician aligns the tip T of the actual needle 1 in the live 2D camera image with the projected needle insertion point q2D to set the insertion point and the end of the needle with the projected needle end point u2D to set the needle direction.

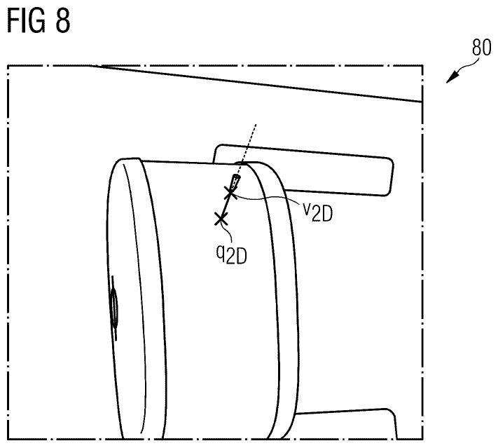

In FIG. 8, a virtual image 80 of a needle 1 including the planned needle end position is shown. The planned needle end position v is visualized by the projected planned needle end position V2D to indicate to the user when to stop the insertion as the planned target position p of the tip of the needle (not shown) is reached. Further, also the projected insertion point q2D is visualized in FIG. 8. The initial needle orientation is drawn as a dashed line.

The visualization may also comprise information about the target point p where the needle shall stop inside the body. The tip of the needle obviously cannot be seen as it approaches the target point p inside the body. However, the visualization on the outside of the body can indicate where the end of the needle shall be when the tip is at the target. Therefore, the end point vCT of the needle head (at the start of the intervention) is moved in needle direction according to formula (7). Then, the position v2D of the end point vCT of the needle head at the end of the intervention in 2D camera image data is calculated according to formula (8). A visual indication like a dot is then placed at this position to indicate the stop position for the needle end point in the live 2D camera image, as illustrated in FIG. 8.

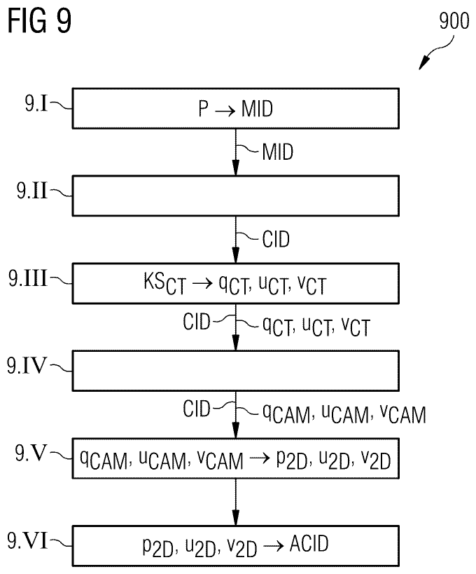

In FIG. 9, a flow chart 900 is shown, illustrating the camera-based method for determining information for guiding a needle intervention.

In step 9.I, medical image data MID of an examination portion of a patient P are received.

In step 9.II, camera image data CID of the examination portion are received.

In step 9.III, an planned insertion position qCT, a needle end point uCT during the start of insertion of the needle 1 and a planned needle end position vCT are determined in the received medical image data MID in the medical image data coordinate system KSCT. As mentioned above, the template for the planned insertion of a needle is preferably determined such that the violation of organs is avoided and the examination portion is hit by the needle.

In step 9. IV, the coordinates qCT, uCT, VCT assigned to the planned insertion pose of the needle 1 are transformed from the medical image data coordinate system KSCT into a 3D camera coordinate system KSCAM.

In step 9.V, the transformed coordinates qCAM, uCAM, VCAM are projected into a 2D camera image coordinate system and 2D coordinates p2D, u2D, v2D assigned to the planned insertion pose are determined.

In step 9.VI, augmented camera image data ACID are generated based on the determined 2D coordinates p2D, u2D, v2D and the camera image data CID.

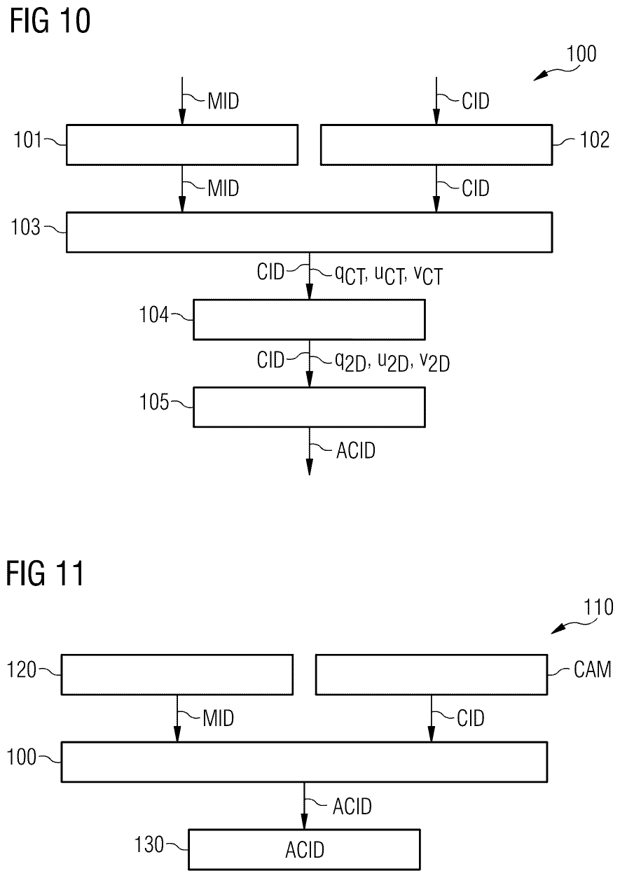

In FIG. 10, a schematic view of an intervention control device 100 according to an embodiment of the present invention is shown.

The intervention control device 100 comprises a medical image data interface 101 for receiving medical image data MID of an examination portion of a patient.

Further, the intervention control device 100 comprises a camera image data interface 102 for receiving camera image data CID of the examination portion.

Furthermore, the intervention control device 100 comprises a determination unit 103 for determining information concerning an insertion pose of the needle 1 in the received medical image data MID, wherein coordinates qCT, uCT, vCT assigned to the insertion pose of the needle 1 in the medical image data coordinate system KSCT are determined.

The intervention control device 100 also includes a transformation unit 104 for transforming the coordinates qCT, uCT, vCT assigned to the insertion pose of the needle 1 in the medical image data coordinate system KSCT into 2D coordinates q2D, u2D, v2D assigned to a 2D view plane of the camera.

The intervention control device 100 further comprises a generation unit 105 for generating augmented camera image data ACID based on the determined coordinates q2D, u2D, v2D in the camera coordinate system KSCAM and based on the received camera image data CID.

In FIG. 11, an intervention system 110 comprising a medical imaging system 120 is shown. For example, the medical imaging system 120 comprises a CT system. The medical imaging system 120 is used for generating medical image data MID from an examination portion of a patient. It is planned to insert a needle into an insertion portion and further to an examination portion of a patient for carrying out an intervention in the examination portion. For identifying and localizing the insertion portion and the examination portion, the medical image data MID are used. Further, the intervention system 110 comprises an observation camera CAM. The camera CAM is used for observing the patient and in particular the insertion portion by recording camera image data CID. The intervention system 110 also comprises a needle 1 for intervention (not shown in FIG. 11). Furthermore, the intervention system 110 comprises an intervention control device 100 as illustrated in FIG. 10 for controlling the intervention using the needle 1. The intervention is performed based on augmented camera image data generated by the intervention control device 100.

Further, the intervention system 110 comprises a display 130 for displaying the augmented camera image data ACID the needle 1 and for displaying a planned pose of the needle 1 based on determined coordinates p2D, u2D, v2D provided by the intervention control device 100.

Finally, it should be pointed out once again that the detailed methods and structures described above are exemplary embodiments and that the basic principle can also be varied in wide areas by the person skilled in the art without leaving the scope of the present invention, insofar as it is specified by the claims. For the sake of completeness, it should also be noted that the use of the indefinite articles “a” or “an” does not exclude the fact that the characteristics in question can be present multiple times. Likewise, the term “unit” does not exclude the fact that it consists of several components, which may also be spatially distributed. Further, independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for generating augmented camera image data for guiding a needle intervention, the computer-implemented method comprising:
receiving medical image data of an examination portion of a patient;
receiving camera image data of the examination portion, the camera image data acquired using a camera;
determining information concerning a planned insertion pose of a needle based on the medical image data, the information including coordinates assigned to the planned insertion pose of the needle in a medical image data coordinate system;
determining coordinates assigned to the planned insertion pose of the needle in a camera coordinate system of the camera by transforming the coordinates assigned to the planned insertion pose of the needle in the medical image data coordinate system into the camera coordinate system; and
generating augmented camera image data based on the camera image data and the coordinates assigned to the planned insertion pose of the needle in the camera coordinate system.

2. The computer-implemented method according to claim 1, wherein the information concerning the planned insertion pose of the needle includes a needle insertion point.

3. The computer-implemented method according to claim 2, wherein
the information concerning the planned insertion pose of the needle includes a needle orientation for the needle intervention, and
the determining information concerning the planned insertion pose of the needle in the medical image data includes determining at least one of (i) an end point of the needle at the start of the insertion based on a target needle tip position, (ii) a planned needle direction, or (iii) a length of the needle.

4. The computer-implemented method according to claim 3, wherein the end point of the needle at the start of the insertion is determined by virtually moving the needle insertion point in an opposite direction of a planned needle direction.

5. The computer-implemented method according to claim 1, wherein the information concerning the planned insertion pose of the needle in the medical image data comprises a planned end point of a head of the needle at the end of the needle intervention.

6. The computer-implemented method according to claim 5, wherein the planned end point of the head of the needle at the end of the needle intervention is determined by addition of a target needle tip position to the end point of the needle at the start of the insertion and subtracting a needle insertion point.

7. The computer-implemented method according to claim 1, wherein
the camera image data includes 2D camera image data, and
the determining coordinates assigned to the planned insertion pose of the needle in the camera coordinate system of the camera includes
transforming the coordinates assigned to the planned insertion pose from the medical image data coordinate system into a 3D camera coordinate system, and
projecting the transformed coordinates into 2D camera image data to determine 2D coordinates assigned to the planned insertion pose.

8. The computer-implemented method according to claim 7, wherein the transforming the coordinates assigned to the planned insertion pose from the medical image data coordinate system into a 3D camera coordinate system includes applying a camera transform to the coordinates assigned to the planned insertion pose in the medical image data coordinate system.

9. The computer-implemented method according to claim 7, wherein the transforming the coordinates assigned to the planned insertion pose from the medical image data coordinate system into a 3D camera coordinate system includes applying a transformation representing a new table position.

10. The computer-implemented method according to claim 8, wherein the camera transform is determined based on an extrinsic calibration step.

11. A camera-based method for guiding a needle intervention, the camera-based method comprising:
generating augmented camera image data by performing the computer-implemented method according to claim 1; and
displaying the augmented camera image data.

12. An intervention control device, comprising:
a medical image data interface to receive medical image data of an examination portion of a patient;
a camera image data interface to receive camera image data of the examination portion;
a determination unit to determine information concerning a planned insertion pose of a needle based on the medical image data, the information including coordinates assigned to the planned insertion pose of the needle in a medical image data coordinate system;
a transformation unit to determine coordinates assigned to the planned insertion pose of the needle in a camera coordinate system of a camera by transforming the coordinates assigned to the planned insertion pose from the medical image data coordinate system into the camera coordinate system; and
a generation unit to generate augmented camera image data based on the camera image data and the coordinates assigned to the planned insertion pose of the needle in the camera coordinate system.

13. An intervention system, comprising:
a medical imaging system;
a camera;
a needle for an intervention;
an intervention control device according to claim 12, wherein the intervention control device is configured to generate augmented camera image data based on medical image data from the medical imaging system and camera image data from the camera, and wherein the intervention control device is configured to control an intervention using the needle based on the augmented camera image data; and
a display to display the augmented camera image data generated by the intervention control device.

14. A non-transitory computer program product comprising instructions that, when executed by a computer, cause the computer to perform the computer-implemented method of claim 1.

15. A non-transitory computer-readable storage medium comprising instructions that, when executed by a computer, cause the computer to perform the computer-implemented method of claim 1.

16. The computer-implemented method according to claim 3, wherein the information concerning the planned insertion pose of the needle in the medical image data comprises a planned end point of a head of the needle at the end of the needle intervention.

17. The computer-implemented method according to claim 16, wherein the planned end point of the head of the needle at the end of the needle intervention is determined by addition of the target needle tip position to the end point of the needle at the start of the insertion and subtracting the needle insertion point.

18. The computer-implemented method according to claim 3, wherein
the camera image data includes 2D camera image data, and
the determining coordinates assigned to the planned insertion pose of the needle in the camera coordinate system of the camera includes
transforming the coordinates assigned to the planned insertion pose from the medical image data coordinate system into a 3D camera coordinate system, and
projecting the transformed coordinates into 2D camera image data to determine 2D coordinates assigned to the planned insertion pose.

19. The computer-implemented method according to claim 8, wherein the transforming the coordinates assigned to the planned insertion pose from the medical image data coordinate system into a 3D camera coordinate system includes applying a transformation representing a new table position.

20. An intervention control device, comprising:
a medical image data interface to receive medical image data of an examination portion of a patient;
a camera image data interface to receive camera image data of the examination portion; and
at least one processor configured to
determine information concerning a planned insertion pose of a needle based on the medical image data, the information including coordinates assigned to the planned insertion pose of the needle in a medical image data coordinate system,
determine coordinates assigned to the planned insertion pose of the needle in a camera coordinate system of the camera by transforming the coordinates assigned to the planned insertion pose from the medical image data coordinate system into the camera coordinate system, and
generate augmented camera image data based on the camera image data and the coordinates assigned to the planned insertion pose in the camera coordinate system.
